(19) United States
(12) Patent Application Publication
(10) Pub. No.: US 2021/0164885 A1
(43) Pub. Date: Jun. 3, 2021

(54) METHOD FOR DETECTING A BLOOD SAMPLE, BLOOD CELL ANALYZER, AND STORAGE MEDIUM

(71) Applicants: SHENZHEN MINDRAY BIO-MEDICAL ELECTRONICS CO., LTD., Shenzhen (CN); SHENZHEN MINDRAY SCIENTIFIC CO., LTD., Shenzhen (CN)

(72) Inventors: Bo YE, Shenzhen (CN); Wenbo ZHENG, Shenzhen (CN); Jin LI, Shenzhen (CN); Huan QI, Shenzhen (CN); Ruiteng TANG, Shenzhen (CN)

(21) Appl. No.: 17/170,779

(22) Filed: Feb. 8, 2021

Related U.S. Application Data
(63) Continuation of application No. PCT/CN2018/103066, filed on Aug. 29, 2018.

(51) Int. Cl. G01N 15/14; G01N 21/64; G01N 33/50

(52) U.S. Cl. CPC G01N 15/1436; G01N 15/1459; G01N 2015/0065; G01N 21/6428; G01N 33/5094; G01N 15/147

(57) ABSTRACT

Disclosed are a method for detecting a blood sample, a blood cell analyzer, and a storage medium. The method includes: acquiring at least two types of optical signal values of cells in a sample from a target detection channel, and generating a scattergram or a data array according to the at least two types of optical signal values of the cells; detecting cell distribution information of an aging characteristic region in the scattergram or data array; and outputting a detection result according to the cell distribution information.

### CROSS-REFERENCE TO RELATED APPLICATIONS

This application is a continuation of PCT Application No. PCT/CN2018/103066, filed Aug. 29, 2018, for BLOOD SAMPLE TESTING METHOD, BLOOD SAMPLE TESTING DEVICE, AND STORAGE MEDIUM, which is incorporated herein by reference.

### TECHNICAL FIELD

The present application relates to the field of blood analysis, and in particular relates to a method for detecting a blood sample, a blood cell analyzer, and a storage medium.

### BACKGROUND

A blood cell analyzer is used to detect distribution information of cells in blood. Blood cells include white blood cells, red blood cells, and platelets. Cell distribution information detected by the blood cell analyzer is generally used for screening and diagnosis of infectious diseases, blood system diseases, autoimmune diseases, and blood coagulation disorders. Thus, it is broadly significant in clinical settings to accurately discover changes in cell distribution information (cell number, cell morphology, etc.).

In recent years, with the widespread use of blood cell analyzers in clinics, after analyzing cells in venous blood samples, the samples must be stored in a laboratory for some days before it can be treated as medical waste of the laboratory, so as to provide the clinic with opportunities for reexamination, check and error correction. In some countries and regions, blood samples are transported to large central laboratories for detecting, and the transportation usually takes at least one day. Accordingly, the accuracy of detection results should be guaranteed after the samples are stored under certain conditions for a certain time period.

A large number of literature studies have shown that as the samples are stored under different conditions for different time periods, cell parameters related to cell distribution detected by blood cell analyzers change significantly due to changes of blood cells themselves, and the samples thus become aged samples. If users directly adopt detection results of the aged samples, the accuracy of the detection results cannot be guaranteed.

### SUMMARY

Accordingly, in order to solve at least one of the above technical problems, there is provided a method for detecting a blood sample, a blood cell analyzer and a storage medium that can determine whether a sample is an aged sample.

According to one aspect, a method for detecting a blood sample may including acquiring at least two types of optical signal values of cells in a sample from a target detection channel, and generating a scattergram or a data array according to the at least two types of optical signal values of the cells; detecting cell distribution information of an aging characteristic region in the scattergram or data array; and outputting a detection result according to the cell distribution information.

In one embodiment, outputting a detection result according to the cell distribution information includes: judging whether the sample is an aged sample according to the cell distribution information, and outputting the detection result according to a judgment result.

In one embodiment, the cell distribution information includes a number of cells in the aging characteristic region; and judging whether the sample is an aged sample according to the cell distribution information includes: judging that the sample is an aged sample if a number of cells in the aging characteristic region is greater than a first preset threshold.

In one embodiment, the cell distribution information includes a ratio of a number of cells in the aging characteristic region to a number of white blood cells in the scattergram or data array, or a ratio of a number of cells in the aging characteristic region to a number of white blood cells of a specified type in the scattergram or data array, wherein the white blood cells of the specified type are preferably neutrophils; and judging whether the sample is an aged sample according to the cell distribution information includes: judging that the sample is an aged sample if the ratio is greater than a second preset threshold.

In one embodiment, outputting the detection result according to the judgment result includes providing an alarm or a prompt if the sample is judged to be an aged sample.

In one embodiment, outputting a detection result according to the cell distribution information includes determining an aging time or aging degree of the sample according to the cell distribution information, and outputting the detection result according to the aging time or aging degree of the sample.

In one embodiment, the method further includes providing an alarm or a prompt according to the aging time or aging degree of the sample.

In one embodiment, the cell distribution information includes a number of cells in the aging characteristic region, and wherein a number of cells in the aging characteristic region is positively correlated with the aging time or aging degree of the sample.

In one embodiment, the cell distribution information includes a ratio of a number of cells in the aging characteristic region to a number of white blood cells in the scattergram or data array, or a ratio of a number of cells in the aging characteristic region to a number of white blood cells of a specified type in the scattergram or data array, wherein the white blood cells of the specified type are preferably neutrophils; and the ratio is positively correlated with the aging time or aging degree of the sample.

In one embodiment, determining an aging time or aging degree of the sample according to the cell distribution information includes: determining a characteristic aging index corresponding to the cell distribution information according to a first predetermined function relationship between the cell distribution information and the characteristic aging index, wherein the characteristic aging index is used to indicate the aging time or aging degree of the sample.

In one embodiment, outputting the detection result according to the aging time or aging degree of the sample includes correcting a cell parameter of the sample according to the characteristic aging index.

In one embodiment, the method further includes determining a characteristic correction coefficient for the cell parameter of the sample according to the characteristic aging index and a second predetermined function relationship between the characteristic aging index and the characteristic correction coefficient, and correcting the cell parameter of the sample according to the characteristic correction coefficient, wherein the characteristic correction coefficient is used to indicate a correction degree of the cell parameter.

In one embodiment, outputting a detection result according to the cell distribution information includes correcting a cell parameter of the sample according to the cell distribution information.

In one embodiment, the cell parameter includes at least one of mean platelet volume, mean corpuscular volume, hematocrit, and red cell volume distribution width.

In one embodiment, acquiring at least two types of optical signal values of cells in the sample from a target detection channel includes: acquiring forward scattered light values and side scattered light values of cells in the sample from the target detection channel; and the at least two types of optical signal values include the forward scattered light values and the side scattered light values.

In one embodiment, the method further includes classifying or counting white blood cells according to the forward scattered light values and the side scattered light values of the cells.

In one embodiment, the method further includes acquiring fluorescent signals of the sample; and the at least two types of optical signal values include the forward scattered light values and the side scattered light values, or the at least two types of optical signal values include the side scattered light values and fluorescence intensity values.

In one embodiment, the method further includes acquiring fluorescent signals of the sample; and classifying or counting white blood cells according to the side scattered light values and fluorescence intensity values of the cells.

In one embodiment, the method further includes acquiring fluorescent signals of the sample; and counting white blood cells, identifying nucleated red blood cells or classifying basophils according to the forward scattered light values and fluorescence intensity values of the cells.

In one embodiment, the aging characteristic region is a region determined based on a white blood cell particle population region in the scattergram.

In one embodiment, the aging characteristic region at least includes a side region with small side scattered light values in the white blood cell particle population region in the scattergram.

In one embodiment, the aging characteristic region includes at least part of a region between the white blood cell particle population and a ghost particle population in the scattergram.

In one embodiment, the aging characteristic region is at least part of a region in the scattergram where the side scattered light values are smaller than a set threshold.

In one embodiment, outputting a detection result according to the cell distribution information includes providing an alarm or a prompt on a user interface according to the cell distribution information; or displaying a corrected cell parameter of the sample on a user interface.

According to another aspect, a blood cell analyzer includes: at least one reaction chamber, configured to provide a reaction place for a sample and a reagent; an optical detection device, configured to irradiate the sample treated with the reagent, collect optical signals generated by each particle in the sample treated with the reagent due to the irradiation, and convert the optical signals into electrical signals to output optical signal information; a delivery device, configured to deliver the sample treated with the reagent from the reaction chamber to the optical detection device; and a processor, configured to receive and process the optical signal information outputted by the optical detection device to obtain detection parameters of the sample; wherein the processor acquires at least two types of optical signal values of cells in the sample from a target detection channel, and generates a scattergram or a data array according to the at least two types of optical signal values of the cells; detects cell distribution information of an aging characteristic region in the scattergram or data array; and outputs a detection result according to the cell distribution information.

In one embodiment, the processor is configured to judge whether a sample is an aged sample according to the cell distribution information, and output a detection result according to a judgment result.

In one embodiment, the cell distribution information includes a number of cells in the aging characteristic region; and the processor is configured to judge that the sample is an aged sample if the number of cells in the aging characteristic region is greater than a first preset threshold.

In one embodiment, the cell distribution information includes: ratio of the number of cells in the aging characteristic region to the number of white blood cells in the scattergram or data array, or ratio of the number of cells in the aging characteristic region to the number of white blood cells of a specified type in the scattergram or data array, wherein the white blood cells of the specified type are preferably neutrophils; and the processor is configured to judge that the sample is an aged sample if the ratio is greater than a second preset threshold.

In one embodiment, the blood cell analyzer further includes a prompt module; and the processor is configured to control the prompt module to provide an alarm or a prompt.

In one embodiment, the processor is further configured to determine an aging time or aging degree of the sample according to the cell distribution information.

In one embodiment, the processor is further configured to correct a cell parameter of the sample according to the cell distribution information.

In one embodiment, the cell parameter includes at least one of mean platelet volume, mean corpuscular volume, hematocrit, and red cell volume distribution width.

In one embodiment, the processor is configured to acquire forward scattered light values and side scattered light values of the cells in the sample from the target detection channel.

In one embodiment, the processor is further configured to classify or count white blood cells according to the forward scattered light values and the side scattered light values of the cells.

In one embodiment, the processor is further configured to acquire fluorescent signals of the sample; and the at least two types of optical signal values include the forward scattered light values and the side scattered light values, or the at least two types of optical signal values include the side scattered light values and fluorescence intensity values.

In one embodiment, the processor is further configured to acquire fluorescent signals of the sample; and classify white blood cells according to the side scattered light values and fluorescence intensity values of the cells.

In one embodiment, the processor is further configured to acquire fluorescent signals of the sample; and count white blood cells, identify nucleated red blood cells or classify basophils according to the forward scattered light values and fluorescence intensity values of the cells.

In one embodiment, the blood cell analyzer further includes a display; and the processor is configured to control a user interface of the display to provide an alarm or a prompt on the user interface according to the cell distribution information; or display a corrected cell parameter of the sample on the user interface.

A computer-readable storage medium storing computer programs thereon, wherein the computer programs, when executed by a processor, implement the steps of the method according to any of the above embodiments.

Through creative efforts, the applicant discovered that cell distribution of a specific region (i.e., aging characteristic region) in the cell scattergram or data array is related to the aging of the sample, and thus proposes to output the detection result based on the cell distribution information of the aging characteristic region, for example for aging identification, thereby ensuring the accuracy of the sample detection result.

### BRIEF DESCRIPTION OF THE DRAWINGS

FIG. 1 is a flowchart of a method according to an embodiment of the present application;

FIG. 2 is a schematic structural diagram of a flow cytometer according to an embodiment of the present application;

FIG. 3A is a two-dimensional SSC-FSC scattergram;

FIG. 3B is a two-dimensional SSC-SFL scattergram;

FIG. 3C is a two-dimensional FSC-SFL scattergram;

FIG. 4 is a schematic diagram of aging characteristic regions of two-dimensional SSC-FSC scattergrams under different aging indexes;

FIG. 5 is a schematic diagram of aging characteristic regions of three-dimensional SSC-FSC-SFL scattergrams under different aging indexes;

FIG. 6 is a schematic diagram of a relationship between cell distribution information of aging characteristic region and aging index;

FIG. 7 is a schematic diagram of a function relationship model between aging index and correction coefficient of MCV;

FIG. 8 is a schematic diagram of a function relationship model between aging index and correction coefficient of RDW-SD;

FIG. 9 is a schematic comparison diagram of MCV mean values of N samples before and after correction;

FIG. 10 is a schematic comparison diagram of RDW_SD mean values of N samples before and after correction;

FIG. 11 is a schematic comparison diagram of MCV values of sample 1 before and after correction;

FIG. 12 is a schematic comparison diagram of RDW_SD values of sample 1 before and after correction;

FIG. 13 is a schematic comparison diagram of MCV values of sample 2 before and after correction;

FIG. 14 is a schematic comparison diagram of RDW_SD values of sample 2 before and after correction;

FIG. 15 is a schematic structural diagram of a device according to an embodiment of the present application; and

FIG. 16 is a schematic structural diagram of an equipment according to an embodiment of the present application.

### DESCRIPTION OF THE EMBODIMENTS

In order to make the objectives, technical solutions, and advantages of the present application clearer, the present application is further described in detail below in combination with the accompanying drawings and embodiments. It should be appreciated that the specific embodiments described herein are only used for interpreting the present application, rather than limiting the present application.

When a cell analysis device is used to analyze a sample (for example, a blood sample), the sample is first treated with reagents, and then optical signal values of cells in the sample are detected to obtain various scattergrams, and at least one cell parameter of the sample can be obtained by analyzing the scattergrams, for example, particle information of white blood cell particle population can be obtained. After analyzing the scattergrams of a large number of samples stored at room temperature for different time periods, the applicant has found that the particle population in a specific region of a scattergram has a strong correlation with the storage conditions (generally temperature and time period) of the samples. Taking a scattergram of a white blood cell channel as an example, it has been found through intensive studies that the particle population in the specific region is a large-volume white blood cell particle population (mainly a neutral particle population).

The optical signal values may include, but are not limited to: forward scattered light values (e.g., forward scattered light intensity, FSC), side scattered light values (e.g., side scattered light intensity, SSC), and light absorption values that characterize nucleic acid content (e.g., fluorescence intensity, SFL). Correspondingly, the applicant has found that there is a particle population, which is related to sample storage conditions, existing in a specific region of each of a two-dimensional SSC-FSC scattergram, a three-dimensional SSC-FSC-SFL scattergram, and a two-dimensional SSC-SFL scattergram, and the two-dimensional SSC-FSC scattergram is particularly typical.

The cell parameter may include red blood cell parameters, white blood cell parameters and platelet parameters, and may be, but are not limited to at least one of mean platelet volume, mean corpuscular volume, hematocrit, red cell volume distribution width, reticulocyte ratio and neutrophil percentage.

Through analysis, the applicant believes that a large-volume white blood cell particle population in the specific region of the scattergram of the white blood cell channel because cell membrane permeability changes with the aging of blood sample, and after being treated with the reagents in the blood cell analyzer, cell membrane is partially damaged, and cytoplasm in cells overflows, so SSC signals that characterize intracellular granularity become small, and FSC signals that characterize cell volume also become small. A small-volume white blood cell particle population will not appear in the specific region because small-volume white blood cells (lymph particles LYM) have very little cytoplasm, and mainly include cell nucleus inside, so SSC signals change little, and FSC signals that characterize cell volume also change little.

It should be noted that, for ease of understanding, the present application only takes distribution characteristics of an aged sample in the scattergram of the white blood cell channel as an example for analysis and explanation. Based on the applicant's creative efforts, it has been found through research and analysis that the cell distribution of a specific region in the scattergram correlates with the aging of samples. Therefore, if there is a correlation between cell distribution of a specific region in a scattergram of another detection channel and the aging of samples, the method according to the embodiments of the present application is also applicable.

In the embodiments of the present application, an aged sample is relative to a fresh sample. After a collected fresh sample has been stored under different storage conditions, cells change, and such a sample is referred to as the aged sample.

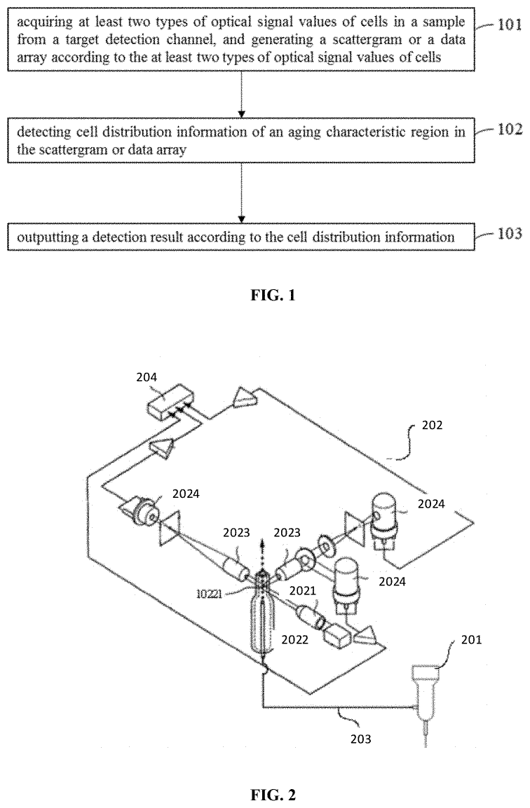

Based on the above research and analysis results, an embodiment of the present application provides a method for detecting a blood sample, which is applied to a blood cell analyzer or a cell analysis device (for example, a flow cytometer), and as shown in FIG. 1, includes the following steps:

Step 101, acquiring at least two types of optical signal values of cells in a sample from a target detection channel, and generating a scattergram or a data array according to the at least two types of optical signal values of cells.

Herein, the target detection channel may be but is not limited to a white blood cell channel.

As described above, the at least two types of optical signal values include forward scattered light values and side scattered light values; or, the at least two types of optical signal values include side scattered light values and light absorption values that characterize nucleic acid content; or, the at least two types of optical signal values include forward scattered light values, side scattered light values, and light absorption values that characterize nucleic acid content. Correspondingly, the generated scattergram may be a two-dimensional scattergram or a three-dimensional scattergram. In addition, in some embodiments, the scattergram is not presented in a graphical form, but is presented in the form of a data array (for example, a two-dimensional data array) of optical signal values. In the following description, the scattergram is taken as an example.

Step 102, detecting cell distribution information of an aging characteristic region in the scattergram or data array.

Herein, the aging characteristic region is a region in the scattergram where a particle population related to the aging of the sample appears. In this embodiment, the cells mapped to the scattergram are called particles, and cells of the same type are aggregated in the scattergram due to their similar optical signal characteristics, and thus called a particle population.

The cell distribution information refers to information that reflects a number of cells in the aging characteristic region, and may be the number of cells, cell distribution area, cell distribution width, etc.

Step 103, outputting a detection result according to the cell distribution information.

The detection result may be outputted according to the cell distribution information in multiple implementation modes. For example, the cell distribution information of the aging characteristic region in the scattergram may be outputted; whether the sample is an aged sample may be judged according to the cell distribution information, and then the detection result is outputted according to a judgment result; an aging time or aging degree of the sample may be determined according to the cell distribution information; and a cell parameter of the sample may be corrected according to a characteristic aging index, which will be explained in detail below. Of course, the foregoing implementation modes may be combined in any way. In this way, the cell distribution information can not only be used to judge whether the sample is an aged sample, but can also be directly used to prompt users of the aging time or aging degree, and can be used to correct the cell parameter of the sample without judging whether the sample is an aged sample. Therefore, if there are a large number of samples, samples that need to be corrected can be directly corrected without judging whether the samples are aged samples. The aging degree refers to the degree to which the sample changes with the environment (e.g., temperature, humidity, etc.) or time.

In some embodiments, outputting a detection result according to the cell distribution information may include outputting the cell distribution information of the aging characteristic region in the scattergram, for example, outputting a number of cells in the aging characteristic region or a number of cells of a specified type, or outputting ratio of a number of cells in the aging characteristic region to a number of cells in the scattergram, or outputting ratio of a number of cells in the aging characteristic region to a number of cells of a specified type in the scattergram.

In some embodiments, outputting a detection result according to the cell distribution information includes judging whether the sample is an aged sample according to the cell distribution information and outputting a detection result according to a judgment result. In some embodiments, the cell parameter of the sample may also be corrected according to the cell distribution information. After the cell parameter is corrected, the detection result is outputted. Also, in some embodiments, whether the sample is an aged sample is judged according to the cell distribution information, and the detection result is outputted according to the judgment result, for example, a prompt or an alarm is provided to users. At the same time, the cell parameter of the sample may further be corrected according to the cell distribution information.

The corrected cell parameter may include red blood cell parameters, white blood cell parameters and platelet parameters, and may be, but are not limited to at least one of mean platelet volume, mean corpuscular volume, hematocrit, red cell volume distribution width, reticulocyte ratio and neutrophil percentage.

The method provided by the embodiments of the present application can be performed during cell analysis and test (such as classification or counting of white blood cells, classification of basophils, and identification of nucleated red blood cells), without a separate processing, thereby simplifying the processing and improving the processing efficiency. Specifically, step 101 above is a step of cell analysis and test. After the scattergram is generated, in order to obtain the cell parameter, particle distribution in the scattergram is analyzed, and the cell distribution information of the aging characteristic region can be obtained at the same time during the analysis.

In some embodiments, outputting a detection result according to the cell distribution information includes: determining an aging time or aging degree of the sample according to the cell distribution information. As described above, in the embodiments of the present application, the cell distribution information refers to information that reflects a number of cells in the aging characteristic region. Correspondingly, an implementation mode of determining an aging time or aging degree of the sample according to the cell distribution information may be identifying the aging time or aging degree of the sample according to the number of cells in the aging characteristic region.

Taking the white blood cell channel scattergram as an example, the larger the number of cells in the aging characteristic region is, the longer the aging time of the sample is or the higher the aging degree of the sample is. It should be noted that different aging characteristic regions selected may lead to different basis for judgment. For the same sample, in the same scattergram, the increase in the number of cells in one region inevitably leads to the decrease in the number of cells in another region except this one region. Therefore, in other embodiments, there may be such a situation: the smaller the number of cells in the aging characteristic region is, the longer the aging time of the sample is or the higher the aging degree of the sample is. If the aging identification is performed through scattergrams of another channel, this situation may also occur, which is not limited in the present application. The selection of the aging characteristic region will be mentioned in the following description.

Furthermore, there are many specific ways to present the cell distribution information of the aging characteristic region.

For example, the cell distribution information of the aging characteristic region may be a number of cells in the aging characteristic region. Furthermore, in order to improve the processing accuracy, the cell distribution information of the aging characteristic region may be defined as a number of cells of a specified type in the aging characteristic region. Taking the white blood cell channel scattergram as an example, the cell distribution information of the aging characteristic region may be a number of white blood cells in the aging characteristic region. Correspondingly, a specific implementation mode of the above-mentioned aging identification may be: identifying the aging time or aging degree of the sample according to the number of white blood cells in the aging characteristic region.

Herein, the process of judging whether the sample is an aged sample according to the cell distribution information may include: the sample is an aged sample if the number of cells in the aging characteristic region is greater than a first preset threshold.

As another example, the cell distribution information of the aging characteristic region may be ratio of the number of cells in the aging characteristic region to the number of cells in the scattergram, or ratio of the number of cells in the aging characteristic region to the number of cells of a specified type in the scattergram. Furthermore, in order to improve the processing accuracy, the cell distribution information of the aging characteristic region may also be defined as ratio of the number of cells of a specified type in the aging characteristic region to the number of cells of a specified type in the scattergram.

Taking the white blood cell channel scattergram as an example, the cell distribution information of the aging characteristic region may be ratio of the number of cells in the aging characteristic region to the number of white blood cells in the scattergram. Most of the cells in the aging characteristic region may be considered as white blood cells. Accordingly, the cell distribution information of the aging characteristic region may be ratio of the number of white blood cells in the aging characteristic region to the number of white blood cells in the scattergram. Correspondingly, a specific implementation mode of judging whether the sample is an aged sample according to the cell distribution information may be: identifying the aging time or aging degree of the sample according to the ratio of the number of white blood cells in the aging characteristic region to the number of white blood cells in the scattergram. Alternatively, the cell distribution information of the aging characteristic region is defined as ratio of the number of cells of a specified type in the aging characteristic region to the number of cells of a specified type in the scattergram. Taking the white blood cell channel scattergram as an example, the cell distribution information of the aging characteristic region may be ratio of the number of white blood cells in the aging characteristic region to the number of white blood cells of a specified type (for example, neutrophils, and lymphocytes) in the scattergram, wherein the white blood cells of the specified type are preferably neutrophils. Correspondingly, the above step of aging identification may be: identifying the aging time or aging degree of the sample according to the ratio of the number of white blood cells in the aging characteristic region to the number of neutrophils in the scattergram.

Herein, the process of judging whether the sample is an aged sample according to the cell distribution information may include: the sample is an aged sample if the ratio is greater than a second preset threshold.

Said first preset threshold and said second preset threshold may be obtained by training and statistic of multiple blood samples through experiments. For example, samples with an aging time longer than K (for example, K=8) hours can be judged as aged samples. Said first preset threshold and said second preset threshold may be obtained by performing analysis, training, and statistics on the cell distribution information of the aging characteristic regions in the scattergrams corresponding to these aged samples. Details are not described herein again.

After the sample is judged to be an aged sample, the detection result is output according to the judgment result, for example, an alarm and a prompt may be provided. For example, during measurement of cell parameters of a blood sample, if the sample is judged to be an aged sample, users are prompted that the blood sample is an aged sample, and relevant measurement results may be inaccurate. Of course, if the sample is judged to be an aged sample, an alarm or a prompt can also be provided according to the aging time or aging degree of the sample. For example, an alarm or a prompt is provided after it is determined that the aging time or aging degree is greater than a preset threshold, that is, an alarm or a prompt is provided only when the aging degree reaches a certain degree.

More specifically, the aging time or aging degree of the sample may be represented by a characteristic aging index, a correlation between the cell distribution information of the aging characteristic region and the characteristic aging index is predetermined, and the correlation may be represented by a function (referred to as a first function in the present application), or by a correlation table (the correlation table records one-to-one correspondence between a group of cell distribution information and a group of aging indexes). The present application does not limit the determination way of the correlation. For example, the correlation may be determined by means of simulation, sample training, statistics of a large amount of experimental data.

Taking the first function as an example, correspondingly, a specific implementation mode of determining the aging time or aging degree of the sample according to the cell distribution information may be: determining a characteristic aging index corresponding to the cell distribution information according to the first predetermined function relationship between the cell distribution information and the characteristic aging index. That is, the characteristic aging index corresponding to the cell distribution information is determined according to the acquired cell distribution information and the first function relationship.

Taking the white blood cell channel as an example, the larger the number of cells in the aging characteristic region is, the larger the characteristic aging index is, that is, the number of cells in the aging characteristic region is positively correlated with the aging time or aging degree of the sample.

In some embodiments, outputting a detection result according to the cell distribution information may also include correcting a cell parameter of the sample according to the characteristic aging index.

As described above, in the embodiments of the present application, the cell distribution information may be information that reflects the number of cells in the aging characteristic region. Correspondingly, an implementation mode of correcting the cell parameter of the sample according to the cell distribution information may be: correcting the cell parameter of the sample by reducing according to the number of cells in the aging characteristic region. The correction by reducing the value of the cell parameter. Taking the white blood cell channel as an example, the larger the number of cells in the aging characteristic region is, the larger the degree of the correction by reducing of the cell parameter of the sample is.

There are many specific implementation modes of correcting the cell parameter. For example, the cell parameter may be corrected according to the aging identification result. Specifically, a characteristic correction coefficient for the cell parameter of the sample is determined according to the characteristic aging index, and a second predetermined function relationship between the characteristic aging index and a characteristic correction coefficient, wherein the characteristic correction coefficient is used to indicate a correction degree of the cell parameter; and the cell parameter of the sample is corrected according to the characteristic correction coefficient.

In some embodiments, the cell parameter may also be corrected directly according to the cell distribution information of the aging characteristic region. Specifically, the characteristic correction coefficient for the cell parameter of the sample is determined according to a third predetermined function relationship between the cell distribution information and the cell parameter correction coefficient, and the cell parameter of the sample is corrected according to the characteristic correction coefficient, wherein the cell parameter correction coefficient is used to indicate the correction degree of the cell parameter.

Similarly, the second function relationship or third function relationship may be determined by means of simulation, sample training, and statistics of a large amount of experimental data.

The first function, the second function and the third function will be further described in the following embodiments.

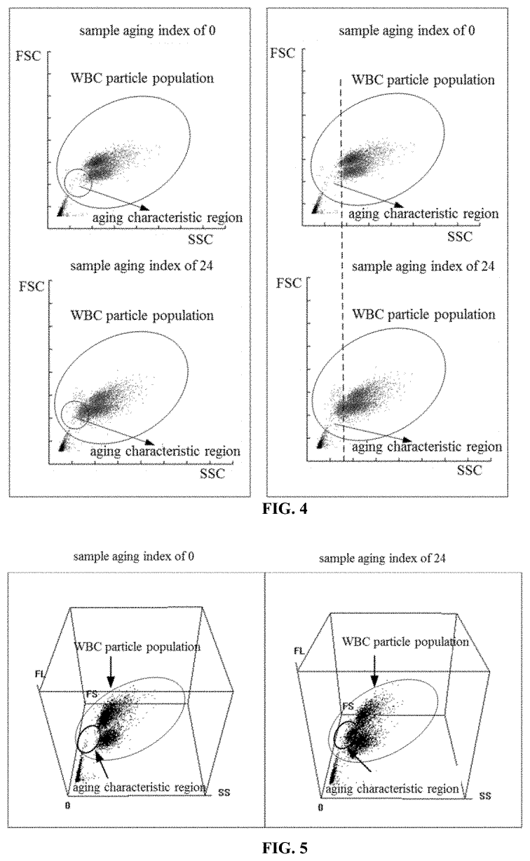

The embodiments of the present application do not limit the determination way of the aging characteristic region. Through tests of a large number of samples, based on simulation analysis or machine learning of the position change data of cell particle populations of the blood samples in scattergrams during the process from normal to aging, the position of the aging characteristic region in the scattergram is determined, or the relative position between the aging characteristic region and a specific particle population is determined. The aging characteristic region may be a fixed region in the scattergram (see circular regions marked in the two scattergrams on the left of FIG. 4 or regions on the left of the dotted line in the two scattergrams on the right of FIG. 4) or a floating region; or a closed region (see the circular regions marked in the two scattergrams on the left of FIG. 4) or an open region (see the regions on the left of the dotted line in the two scattergrams on the right of FIG. 4). Taking the white blood cell channel as an example, the aging characteristic region is a region determined based on a white blood cell particle population region in the scattergram.

For example, the aging characteristic region includes at least one of the following regions:

a particle population edge region with small optical signal values in the white blood cell particle population in the scattergram, for example, a side region with small side scattered light values in the white blood cell particle population region in the scattergram (smaller than a preset value, such as the SSC value represented by the dotted line in the two scattergrams on the left of FIG. 4), or a side region with small side scattered light values and small forward scattered light values in the white blood cell particle population region in the scattergram;

a particle population edge region close to a ghost particle population (for example, the particle population close to the coordinate origin in FIG. 4 and FIG. 5) in the white blood cell particle population in the scattergram;

at least part of a region between the white blood cell particle population and the ghost particle population in the scattergram.

Of course, the aging characteristic region may also be any combination of the above three regions, or other region with aging judgment features, which is not limited herein, and reference may be made to the schematic diagrams of the embodiments in FIGS. 4 and 5.

After the aging identification is performed according to the cell distribution information, the detection result may further be outputted or sent. Herein, the output way may be, but is not limited to, display output, voice broadcast output, a sound and light alarm, etc. “Send” refers to sending to other device, such as a central station, a user's mobile phone terminal, a PC, a server, or a cloud. An alarm or a prompt may be provided, or a corrected cell parameter of the sample may be displayed, or the cell distribution information may be output, on a user interface, for example, an alarm or a prompt for indicating that the sample is an aged sample is provided on the user interface.

Next, a flow cytometer for detecting cells is taken as an example to further illustrate the method according to the embodiments of the present application. Corresponding to the method for detecting a blood sample according to the foregoing embodiments, a blood cell analyzer is provided below, and the blood cell analyzer may be a flow cytometer.

The blood cell analyzer of this embodiment may mainly include the structure shown in FIG. 2: at least one reaction chamber 201, an optical detection device 202, a delivery device 203 and a processor 204, which will be described in detail below.

The reaction chamber 201 is configured to provide a reaction place for a sample and a reagent to prepare a sample solution. Specifically, the blood sample obtained by blood collection may be diluted and labeled with a fluorescent staining reagent to obtain a sample solution. The common fluorescent staining reagent may be pyronine, acridine orange, thiazole orange and the like.

The optical detection device 202 is configured to irradiate the sample treated with the reagent (that is, the above-mentioned sample solution), collect optical signals generated by each particle in the sample treated with the reagent due to the irradiation, and convert the optical signals into electrical signals to output optical signal information (that is, optical signal values). The optical signals here may be forward scattered optical signals (FSC), side scattered optical signals (SSC), and fluorescent scattered optical signals (SFL, referred to herein as fluorescent signals). The optical detection device 202 may include, but is not limited to, a light source 2021 and a sheath flow chamber 2022 with an orifice 20221. The particles in the blood sample can flow in the sheath flow chamber 2022, and pass through the orifice 20221 one by one. Light emitted by the light source 2021 can be irradiated on the particles in the orifice 20221, and scattered optical signals or fluorescent signals are correspondingly generated. The optical detection device 202 may further include lens groups 2023 arranged in front of the orifice and on sides of the orifice respectively, photoelectric sensors 2024 (e.g., photodiodes, photomultiplier tubes) and an A/D converter. The A/D converter may be arranged in the processor 204 or be a separate element, so that the lens groups 2023 can capture the corresponding scattered optical signals and fluorescent signals, the photoelectric sensors 2024 can convert the captured optical signals (referring to scattered optical signals and fluorescent signals, etc.) into electrical signals, and then the A/D converter processes the electrical signals through A/D conversion to obtain digital signals, and the digital signals can be outputted as optical signal information.

The delivery device 203 is configured to deliver the sample (that is, the sample solution) treated with the reagent from the reaction chamber 201 to the optical detection device 202.

The processor 204 is configured to receive and process the optical signal information outputted by the optical detection device 202 to obtain a cell parameter of the sample. Herein, the processor 204 acquires at least two types of optical signal values of cells in the sample from a target detection channel (for example, a white blood cell channel), and generates a scattergram according to the at least two type of optical signal values of the cells; detects cell distribution information of an aging characteristic region in the scattergram; and outputs a detection result according to the cell distribution information. The detection result may include a corrected cell parameter.

In some embodiments, outputting a detection result according to the cell distribution information includes: judging whether the sample is an aged sample according to the cell distribution information, and outputting the detection result according to the judgment result.

The at least two types of optical signal values of cells in the sample acquired by the processor 204 from the target detection channel may be forward scattered light values (FSC) and side scattered light values (SSC). Accordingly, the processor 204 may classify or count white blood cells according to the forward scattered light values and the side scattered light values of the cells. For example, on a user interface, white blood cell classification information may be displayed, and users may be prompted for whether the sample is an aged sample and the aging degree at the same time.

The processor 204 may further acquire fluorescent signals of the sample. In this case, the at least two types of optical signal values include the forward scattered light values and the side scattered light values, or the at least two types of optical signal values include the side scattered light values and fluorescence intensity values (SFL). The processor 204 may classify white blood cells according to the side scattered light values and the fluorescence intensity values of the cells, or count white blood cells, identify nucleated red blood cells or classify basophils according to the forward scattered light values and the fluorescence intensity values of the cells. For example, on the user interface, white blood cell count information and basophil classification information may be displayed, and users may be prompted for whether the sample is an aged sample and the aging degree at the same time. The method for detecting a blood sample and the blood cell analyzer according to the present application can judge whether the sample is an aged sample by means of the aging characteristic region in the scattergram of the forward scattered light values and the side scattered light values of the white blood cell channel, and can further speculate the aging time or aging degree.

In one embodiment, the cell distribution information includes a number of cells in the aging characteristic region; and the processor 204 is configured to judge that the sample is an aged sample if the number of cells in the aging characteristic region is greater than a first preset threshold.

In one embodiment, the cell distribution information includes: ratio of the number of cells in the aging characteristic region to the number of white blood cells in the scattergram, or ratio of the number of cells in the aging characteristic region to the number of white blood cells of a specified type in the scattergram, wherein the white blood cells of the specified type are preferably neutrophils; and the processor 204 is configured to judge that the sample is an aged sample if the ratio is greater than a second preset threshold.

In one embodiment, the blood cell analyzer further includes a prompt module; and the processor 204 is configured to control the prompt module to provide an alarm or a prompt.

In one embodiment, the processor 204 is further configured to determine an aging time or aging degree of the sample according to the cell distribution information.

In one embodiment, the processor 204 is further configured to correct a cell parameter of the sample according to the cell distribution information.

In one embodiment, the cell parameter includes at least one of mean platelet volume, mean corpuscular volume, hematocrit, and red cell volume distribution width.

In one embodiment, the processor 204 is configured to acquire forward scattered light values and side scattered light values of the cells in the sample from the target detection channel.

In one embodiment, the processor 204 is further configured to classify or count white blood cells according to the forward scattered light values and the side scattered light values of the cells.

In one embodiment, the processor 204 is further configured to acquire fluorescent signals of the sample; and the at least two types of optical signal values include the forward scattered light values and the side scattered light values, or the at least two types of optical signal values include the side scattered light values and fluorescence intensity values.

In one embodiment, the processor 204 is further configured to acquire fluorescent signals of the sample; and classify white blood cells according to the side scattered light values and fluorescence intensity values of the cells.

In one embodiment, the processor 204 is further configured to acquire fluorescent signals of the sample; and count white blood cells, identify nucleated red blood cells or classify basophils according to the forward scattered light values and fluorescence intensity values of the cells.

In one embodiment, the blood cell analyzer further includes a display; and the processor 204 is configured to control a user interface of the display to provide an alarm or a prompt, or display the corrected cell parameter of the sample.

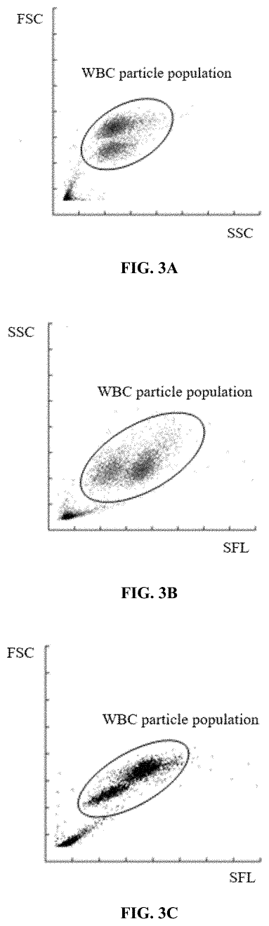

The processor 204 may generate a scattergram by using the detected optical signal values, and obtain a white blood cell particle population (WBC particle population) by analyzing the scattergram. Herein, two-dimensional scattergrams shown in FIGS. 3A to 3C or three-dimensional scattergrams shown in FIG. 4 may be generated.

An aging characteristic region is determined from the two-dimensional SSC-FSC scattergram shown in FIG. 3A. An aging characteristic region may also be determined from a three-dimensional SSC-FSC-SFL scattergram, and an aging characteristic region may further be determined from a two-dimensional SSC-SFL scattergram. It should be noted that, for an abnormal sample (relative to a healthy blood sample), the aging characteristic region of the two-dimensional SSC-SFL scattergram may not be as significant as that of the two-dimensional SSC-FSC scattergram, but can be used for the aging identification or cell parameter correction for the healthy blood sample.

In FIG. 4 and FIG. 5, the aging index is different, and the cell distribution in the characteristic region (i.e., the aging characteristic region) of the scattergram is different.

Hereinafter, some embodiments will be listed for the process in which the processor 204 determines the aging time or aging degree of the sample according to the cell distribution information.

Cell distribution information of the aging characteristic region is acquired, and the cell distribution information is denoted by FeatureCellInfo.

A sample aging index Age_Indice is calculated according to the FeatureCellInfo, wherein the Age_Indice is a function (that is, the first function above) of the FeatureCellInfo:

Age_Indice=f(FeatueCellInfo)

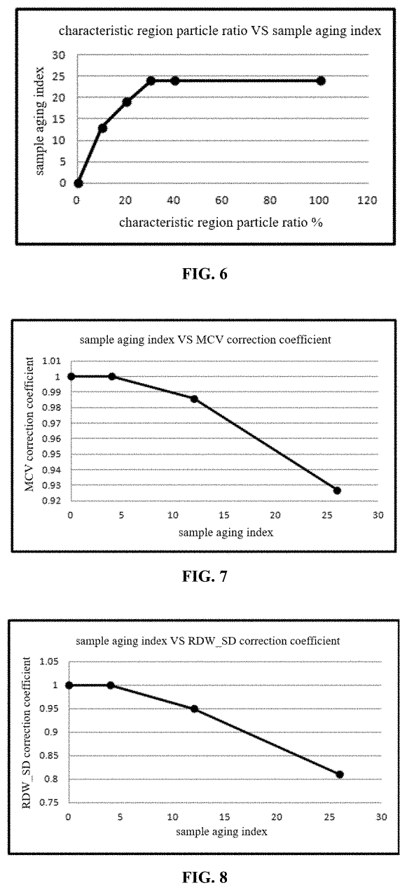

If the aging time or aging degree of the sample is identified according to the ratio of the number of cells in the aging characteristic region to the number of white blood cells in the scattergram, a characteristic aging index corresponding to the sample may be obtained by the following equation 1 (that is, the first function).

$$Y = f ( X ) = \{ \begin{matrix}0 & X \leq 0 \\ 1.3 \times X & 0 < X \leq 10 \\ 0.6 \times X + 7.0 & 10 < X \leq 20 \\ 0.5 \times X + 9.0 & 20 < X \leq 30 \\ 24.0 & X > 30\end{matrix} \qquad ( equation 1 )$$

Where, X is the ratio FeatureCellInfo (which may be abbreviated as a characteristic region particle ratio) of the number of cells in the aging characteristic region to the number of white blood cells in the scattergram, and Y is the aging index Age_Indice. The curve corresponding to equation 1 is shown in FIG. 6.

The detection deviation of the cell parameter of the blood sample is corrected according to the aging index to obtain a final parameter detection result. The corrected result is denoted by Result_E, the result before correction is denoted by Result_F, and the function relationship between the two results is as follows:

Result_E=g(Result_F,T)(second function)

Where, T is Age_Indice, which specifically refers to the aging time or aging degree in this embodiment.

If the corrected cell parameter of the blood sample is mean corpuscular volume MCV, after the characteristic aging index of the sample is obtained according to equation 1, a characteristic correction coefficient corresponding to the sample may be obtained by the following equation 2 (that is, the second function).

$$Y = f ( X ) = \{ \begin{matrix}1.0 & X \leq 4 \\ - 0.00175 \times X + 1.007 & 4 < X \leq 12 \\ - 0.00421 \times X + 1.036571 & 12 < X \leq 26 \\ 0.927 & X > 26\end{matrix} \qquad ( equation 2 )$$

Where, X is the aging index Age_Indice, and Y is the correction coefficient of MCV. The curve corresponding to equation 2 is shown in FIG. 7.

If the corrected cell parameter of the blood sample is red cell volume distribution width RDW_SD (or RDW), after the characteristic aging index of the sample is obtained according to equation 1, a characteristic correction coefficient corresponding to the sample may be obtained by the following equation 3 (that is, the second function).

$$Y = f ( X ) = \{ \begin{matrix}1.0 & X \leq 4 \\ - 0.00625 \times X + 1.025 & 4 < X \leq 12 \\ - 0.01 \times X + 1.07 & 12 < X \leq 26 \\ 0.81 & X > 26\end{matrix} \qquad ( equation 3 )$$

Where, X is the aging index Age_Indice, and Y is the correction coefficient of RDW. The curve corresponding to equation 3 is shown in FIG. 8, wherein RDW_SD is RDW.

After the characteristic correction coefficient is obtained, the characteristic correction coefficient may be multiplied by the cell parameter for correction.

It should be noted that in this embodiment, the final parameter result may also be corrected directly using the ratio of the number of cells in the aging characteristic region in the scattergram to the number of white blood cells in the scattergram, for example:

Result_E=h(FeatureCellRatio,Result_F),

where h is a monotonic function (third function) relative to FeatureCellRatio, and details are not described again.

According to the above process, N blood samples (N=10) were randomly selected for the following test to obtain comparisons of the blood cell parameters before and after correction, after the samples were stored for different time periods:

Each sample was stored at room temperature for 0, 2, 4, 6, 8, 10, 12, 14, 16, 18, 20, 22 and 24 hours, and then detected at each time point on a BC-6000 flow cytometer of Shenzhen Mindray Bio-Medical Electronics Co., Ltd., to obtain the number of cells in the aging characteristic region, the number of white blood cells in the scattergram, and the blood cell parameters (mean corpuscular volume MCV and red cell volume distribution width RDW_SD) of each sample at different time points, and the cell distribution information FeatureCellRatio of the aging characteristic region was calculated according to the number of cells in the aging characteristic region and the number of white blood cells in the scattergram. In this test, the calculation method of FeatureCellRatio is as follows:

$$FeatureCellRatio \% = \frac{the number of particles in the characteristic region}{the total number of white blood cell particles} \times 100 \%$$

Where, the number of particles in the characteristic region is the number of cells in the aging characteristic region, and the total number of white blood cell particles is the number of white blood cells in the scattergram.

The characteristic aging index of the sample is determined according to the FeatureCellRatio (that is, characteristic region particle ratio in FIG. 6). Specifically, the aging index is determined by using a piecewise linear function model shown in FIG. 6. In FIG. 6, the abscissa represents the ratio (that is, characteristic region particle ratio) of the number of cells in the aging characteristic region of the scattergram to the number of white blood cells in the scattergram, and the ordinate represents the characteristic aging index.

The blood cell parameters obtained in this test were mean corpuscular volume MCV and red cell volume distribution width RDW_SD. In this test, the mean corpuscular volume MCV and the red cell volume distribution width RDW_SD were corrected, which is taken as an example to illustrate the correction effect using the embodiments of the present application.

The function relationship between the aging index and the correction coefficient of MCV is shown in FIG. 7, and the function relationship between the aging index and the correction coefficient of RDW_SD is shown in FIG. 8. After the aging index is obtained according to the FeatureCellRatio, the correction coefficients are obtained according to the function relationships between the aging index and the correction coefficients.

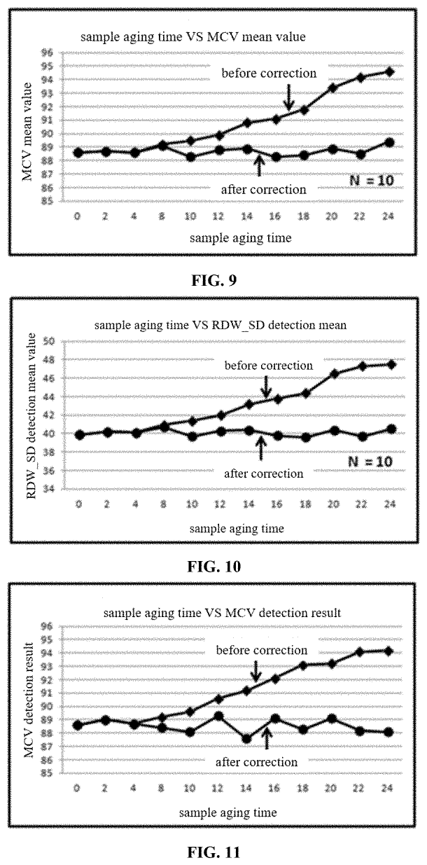

The two parameters MCV and RDW_SD were corrected by using the obtained correction coefficients. The correction results of the N samples and the single sample are shown in FIGS. 9-14. In this test, the aging time is used as the aging index.

FIG. 9 is a schematic comparison diagram of MCV mean values of the N samples before and after correction; and FIG. 10 is a schematic comparison diagram of RDW_SD mean values of the N samples before and after correction. Herein, the abscissa represents the aging time, and the ordinate represents the detection result-blood cell parameter value; the rhombic black dots represent mean blood cell parameters of the N samples before correction, and the circular black dots represent mean blood cell parameters of the N samples after correction.

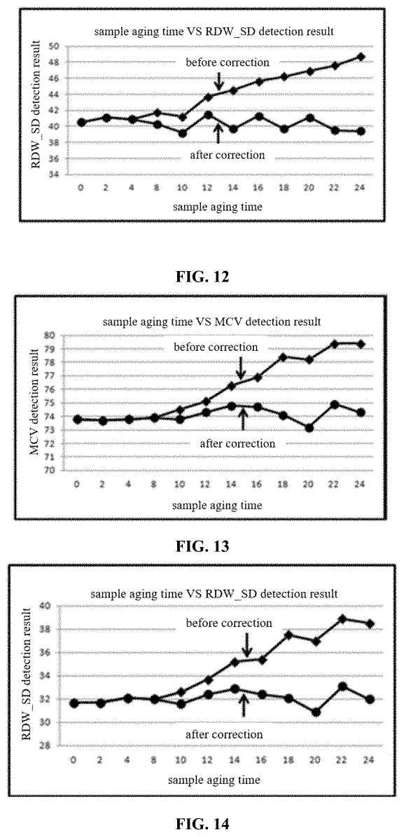

FIG. 11 is a schematic comparison diagram of MCV values of sample 1 before and after correction; and FIG. 12 is a schematic comparison diagram of RDW_SD values of sample 1 before and after correction. Herein, the abscissa represents the aging time, and the ordinate represents the detection result-blood cell parameter value; the rhombic black dots represent blood cell parameters of sample 1 before correction, and the circular black dots represent blood cell parameters of sample 1 after correction.

FIG. 13 is a schematic comparison diagram of MCV values of sample 2 before and after correction; and FIG. 14 is a schematic comparison diagram of RDW_SD values of sample 2 before and after correction. Herein, the abscissa represents the aging time, and the ordinate represents the detection result-blood cell parameter value; the rhombic black dots represent blood cell parameters of sample 2 before correction, and the circular black dots represent blood cell parameters of sample 2 after correction.

According to the figures, fresh blood samples were stored for different storage time, and after the parameters were corrected by the embodiments of the present application, for the deviations in the cell parameter values before aging (for example, the aging time is 0) and after aging, the deviation values (or mean deviations) after correction of the cell parameters were smaller than the deviation values (or mean deviations) before correction. The effects are shown in Tables 1-3 below:

TABLE 1
Mean deviations of blood cell parameters of
the N samples before and after correction

| | Mean deviation of the N samples | |
|---|---|---|
| / | MCV mean deviation | RDW_SD mean deviation of |
| Before correction | 6 | 7.5 |
| After correction | 1.1 | 0.9 |

TABLE 2
Deviation values of blood cell parameters of a single
sample (sample 1) before and after correction

| | Deviation value of sample 1 | |
|---|---|---|
| / | MCV deviation value | RDW_SD deviation value |
| Before correction | 5.6 | 8.2 |
| After correction | 1.7 | 2.3 |

TABLE 3
Deviation values of blood cell parameters of a single
sample (sample 2) before and after correction

| | Deviation value of sample 2 | |
|---|---|---|
| / | MCV deviation value | RDW_SD deviation value |
| Before correction | 5.6 | 7.2 |
| After correction | 1.7 | 2.2 |

The test data diagrams corresponding to Table 1 are shown in FIG. 9 and FIG. 10, the test data diagrams corresponding to Table 2 are shown in FIG. 11 and FIG. 12, and the test data diagrams corresponding to Table 3 are shown in FIG. 13 and FIG. 14. From Table 1, Table 2, Table 3 and the corresponding test data diagrams, it can be seen that for the deviations in the cell parameter values before aging (for example, the aging time is 0) and after aging, the deviation values after correction of the cell parameters were smaller than the deviation values before correction, which proves that after correction, the aging of the samples has less influence on the detection results of MCV and RDW_SD, and the accuracy of parameter detection is significantly improved.

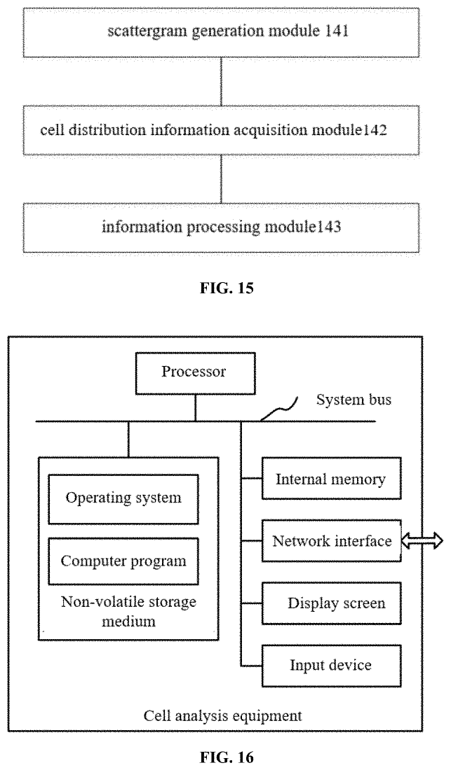

Corresponding to the method for detecting a blood sample in the foregoing embodiments, a device for detecting a blood sample is further provided. In one embodiment, as shown in FIG. 15, a device for detecting a blood sample is provided, including:

a scattergram generation module 141, configured to acquire at least two types of optical signal values of cells in a sample from a target detection channel, and generate a scattergram according to the at least two types of optical signal values of the cells;

a cell distribution information acquisition module 142, configured to detect cell distribution information of an aging characteristic region in the scattergram; and

an information processing module 143, configured to output a detection result according to the cell distribution information. The detection result may be outputted according to the cell distribution information in multiple implementation modes, for example, the cell distribution information of the aging characteristic region in the scattergram may be outputted; whether the sample is an aged sample may be judged according to the cell distribution information, and the detection result may be outputted according to the judgment result; an aging time or aging degree of the sample may be determined according to the cell distribution information; and a cell parameter of the sample may be corrected according to a characteristic aging index, which will be explained in detail below. Of course, the foregoing implementation modes may be combined in any way. For example, the aging identification is performed according to the cell distribution information or the cell parameter of the sample is corrected according to the cell distribution information.

Through creative efforts, the applicant discovered that the cell distribution of a specific region (i.e., aging characteristic region) in the cell scattergram is related to the aging of the sample, and thus proposes to perform aging identification or correct a cell parameter based on the cell distribution information of the aging characteristic region, thereby ensuring the accuracy of the sample detection result.

In one embodiment, the cell distribution information includes the number of cells in the aging characteristic region; and the information processing module 143 is configured to judge that the sample is an aged sample if the number of cells in the aging characteristic region is greater than a first preset threshold.

In one embodiment, the cell distribution information includes: ratio of the number of cells in the aging characteristic region to the number of white blood cells in the scattergram, or ratio of the number of cells in the aging characteristic region to the number of white blood cells of a specified type in the scattergram, wherein the white blood cells of the specified type are preferably neutrophils; and the information processing module 143 is configured to judge that the sample is an aged sample if the ratio is greater than a second preset threshold.

In one embodiment, the device for detecting a blood sample further includes a prompt module (not shown); and the information processing module 143 is configured to control the prompt module to provide an alarm or a prompt.

In one embodiment, the information processing module 143 is further configured to determine an aging time or aging degree of the sample according to the cell distribution information.

In one embodiment, the information processing module 143 is further configured to correct a cell parameter of the sample according to the cell distribution information.

In one embodiment, the cell parameter includes at least one of mean platelet volume, mean corpuscular volume, hematocrit, and red cell volume distribution width.

In one embodiment, the scattergram generation module 141 is configured to acquire forward scattered light values and side scattered light values of the cells in the sample from the target detection channel.

In one embodiment, the information processing module 143 is further configured to classify or count white blood cells according to the forward scattered light values and the side scattered light values of the cells.

In one embodiment, the scattergram generation module 141 is further configured to acquire fluorescent signals of the sample. The at least two types of optical signal values include the forward scattered light values and the side scattered light values, or the at least two types of optical signal values include the side scattered light values and fluorescence intensity values.

In one embodiment, the scattergram generation module 141 is further configured to acquire fluorescent signals of the sample. The information processing module 143 classifies white blood cells according to the side scattered light values and fluorescence intensity values of the cells.

In one embodiment, the scattergram generation module 141 is further configured to acquire fluorescent signals of the sample. The information processing module 143 counts white blood cells, or identifies nucleated red blood cells or classifies basophils according to the forward scattered light values and fluorescence intensity values of the cells.

In one embodiment, the device for detecting a blood sample further includes a display module (not shown); and the information processing module 143 is configured to control a user interface of the display module to provide an alarm or a prompt, or display the corrected cell parameter of the sample.

For specific definitions of the above device, reference can be made to the above definitions of the information processing method, and details are not described herein again. The various modules in the above device may be implemented in whole or in part by software, hardware, and a combination thereof. The above modules may be embedded in or independent of a processor in a computer device in the form of hardware, or stored in a memory of the computer device in the form of software, so that the processor calls and executes the operations corresponding to the modules.

In one embodiment, a cell analysis equipment is provided, and its internal structural diagram may be as shown in FIG. 16 The equipment includes a processor, a memory, a network interface, a display screen, and an input device connected by a system bus. The processor of the equipment is configured to provide computing and control capabilities. The memory of the equipment includes a non-volatile storage medium and an internal memory. The non-volatile storage medium stores an operating system and computer programs. The internal memory provides an environment for the operations of the operating system and the computer programs in the non-volatile storage medium. The network interface of the equipment is configured to communicate with an external terminal through a network connection. When the computer programs are executed by the processor, the above method for detecting a blood sample is implemented. The display screen of the equipment may be a liquid crystal display screen or an electronic ink display screen. The input device of the equipment may be a touch layer covered on the display screen, or a button, trackball or touchpad arranged on a housing of the equipment, or an external keyboard, touchpad or mouse.

Those skilled in the art may appreciate that the structure shown in FIG. 16 is only part of the structure related to the solution of the present application, and does not constitute a limitation to the equipment to which the solution of the present application is applied. The specific equipment may include more or fewer components than those shown in the figure, or combine some components, or have a different component arrangement.

In one embodiment, a cell analysis equipment is provided, including a memory and a processor, the memory stores computer programs, when the computer programs are executed by the processor, the steps of any embodiment of the method for detecting a blood sample above are implemented.

In one embodiment, a computer-readable storage medium storing computer programs thereon is provided, and the computer programs, when executed by a processor, implement the steps of any embodiment of the method for detecting a blood sample above.

A person of ordinary skill in the art may appreciate that all or some of the steps of the method in the above embodiments may be completed by instructing relevant hardware through computer programs. The computer programs may be stored in a non-volatile computer-readable storage medium. The computer programs, when executed, may include the steps of any embodiment of the method above. Any reference to a memory, a storage, a database or other medium used in any embodiment provided by the present application may include a non-volatile memory or a volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. By way of illustration instead of limitation, RAM is available in multiple forms, such as a static RAM (SRAM), a dynamic RAM (DRAM), a synchronous DRAM (SDRAM), a double data rate SDRAM (DDRSDRAM), an enhanced SDRAM (ESDRAM), a Synchlink DRAM (SLDRAM), a Rambus direct RAM (RDRAM), a direct Rambus dynamic RAM (DRDRAM), and a Rambus dynamic RAM (RDRAM).

The technical features of the above embodiments may be combined in any way. For the purpose of simplicity in description, all the possible combinations of the technical features in the above embodiments are not described. However, as long as the combinations of these technical features do not contradict each other, they shall fall within the scope of the specification.

The foregoing embodiments only describe several implementation modes of the present application, and their description is specific and detailed, but cannot therefore be understood as a limitation to the patent scope of the present invention. It should be noted that a person of ordinary skill in the art may further make variations and improvements without departing from the conception of the present application, and these all fall within the protection scope of the present application. Therefore, the patent protection scope of the present application should be subject to the appended claims.

1-39. (canceled)

40. A method for detecting a blood sample, comprising:
acquiring at least two types of optical signal values of cells in a sample from a target detection channel, and generating a scattergram or a data array according to the at least two types of optical signal values of the cells;
detecting cell distribution information of an aging characteristic region in the scattergram or data array; and
outputting a detection result according to the cell distribution information.

41. The method according to claim 40, wherein outputting a detection result according to the cell distribution information comprises: judging whether the sample is an aged sample according to the cell distribution information, and outputting the detection result according to a judgment result.

42. The method according to claim 41, wherein the cell distribution information comprises a number of cells in the aging characteristic region; and
judging whether the sample is an aged sample according to the cell distribution information comprises: judging that the sample is an aged sample if the number of cells in the aging characteristic region is greater than a first preset threshold.

43. The method according to claim 41, wherein the cell distribution information comprises a ratio of a number of cells in the aging characteristic region to a number of white blood cells in the scattergram or data array, or a ratio of a number of cells in the aging characteristic region to a number of white blood cells of a specified type in the scattergram or data array; and
judging whether the sample is an aged sample according to the cell distribution information comprises: judging that the sample is an aged sample if the ratio is greater than a second preset threshold.

44. The method according to claim 40, wherein outputting a detection result according to the cell distribution information comprises:
determining an aging time or aging degree of the sample according to the cell distribution information, and outputting the detection result according to the aging time or aging degree of the sample.

45. The method according to claim 44, wherein the cell distribution information comprises a number of cells in the aging characteristic region, and wherein the number of cells in the aging characteristic region is positively correlated with the aging time or aging degree of the sample.

46. The method according to claim 44, wherein the cell distribution information comprises: a ratio of a number of cells in the aging characteristic region to a number of white blood cells in the scattergram or data array, or a ratio of a number of cells in the aging characteristic region to a number of white blood cells of a specified type in the scattergram or data array; and wherein the ratio is positively correlated with the aging time or aging degree of the sample.

47. The method according to claim 44, wherein determining an aging time or aging degree of the sample according to the cell distribution information comprises:
determining a characteristic aging index corresponding to the cell distribution information according to a first predetermined function relationship between the cell distribution information and the characteristic aging index, wherein the characteristic aging index is used to indicate the aging time or aging degree of the sample.

48. The method according to claim 47, wherein outputting the detection result according to the aging time or aging degree of the sample comprises: correcting a cell parameter of the sample according to the characteristic aging index.

49. The method according to claim 48, wherein correcting a cell parameter of the sample according to the characteristic aging index comprises:
determining a characteristic correction coefficient for the cell parameter of the sample according to the characteristic aging index and a second predetermined function relationship between the characteristic aging index and the characteristic correction coefficient; and correcting the cell parameter of the sample according to the characteristic correction coefficient, wherein the characteristic correction coefficient is used to indicate a correction degree of the cell parameter.

50. The method according to claim 40, wherein outputting a detection result according to the cell distribution information comprises:
correcting a cell parameter of the sample according to the cell distribution information.

51. The method according to claim 50, wherein the cell parameter comprises at least one of mean platelet volume, mean corpuscular volume, hematocrit, and red cell volume distribution width.

52. The method according to claim 40, wherein acquiring at least two types of optical signal values of cells in a sample from a target detection channel comprises: acquiring forward scattered light values and side scattered light values of the cells in the sample from the target detection channel, and
the at least two types of optical signal values comprise the forward scattered light values and the side scattered light values.

53. The method according to claim 40, wherein acquiring at least two types of optical signal values of cells in a sample from a target detection channel comprises: acquiring side scattered light values and fluorescence intensity values of the cells in the sample from the target detection channel, and
the at least two types of optical signal values comprise the side light values and the fluorescence intensity values.

54. The method according to claim 52, wherein the aging characteristic region is a region determined based on a white blood cell particle population region in the scattergram; or
wherein the aging characteristic region is at least part of a region in the scattergram where the side scattered light values are smaller than a preset threshold.

55. The method according to claim 54, wherein the aging characteristic region at least comprises a region with small side scattered light values in the white blood cell particle population region in the scattergram; or
the aging characteristic region comprises at least part of a region between the white blood cell particle population and a ghost particle population in the scattergram.

56. The method according to claim 40, wherein outputting a detection result according to the cell distribution information comprises:
providing an alarm or a prompt on a user interface according to the cell distribution information; or
displaying a corrected cell parameter of the sample on a user interface.

57. A blood cell analyzer, comprising:
at least one reaction chamber, configured to provide a reaction place for a sample and a reagent;
an optical detection device, configured to irradiate the sample treated with the reagent, collect optical signals generated by each particle in the sample treated with the reagent due to the irradiation, and convert the optical signals into electrical signals to output optical signal information;
a delivery device, configured to deliver the sample treated with the reagent from the reaction chamber to the optical detection device; and
a processor, configured to receive and process the optical signal information outputted by the optical detection device to obtain detection parameters of the sample; wherein the processor acquires at least two types of optical signal values of cells in the sample from a target detection channel, and generates a scattergram or a data array according to the at least two types of optical signal values of the cells; detects cell distribution information of an aging characteristic region in the scattergram or data array; and outputs a detection result according to the cell distribution information.

58. The analyzer according to claim 57, wherein the processor is configured to judge whether the sample is an aged sample according to the cell distribution information, and output the detection result according to a judgment result.

59. The analyzer according to claim 57, wherein the processor is configured to determine an aging time or aging degree of the sample according to the cell distribution information.

60. The analyzer according to claim 57, wherein the processor is configured to correct a cell parameter of the sample according to the cell distribution information.
